(19) United States
(12) Patent Application Publication
(10) Pub. No.: US 2026/0232271 A1
(43) Pub. Date: Aug. 13, 2026

(54) TREATMENT AND MONITORING OF DISEASES ASSOCIATED WITH ELEVATED INTRACRANIAL PRESSURE OR HYDROCEPHALUS

(71) Applicant: Biogen MA Inc., Cambridge, MA (US)

(72) Inventors: Daniel J. Abrams, Denver, CO (US); Matias Maturana, Melbourne (AU)

(21) Appl. No.: 17/868,321

(22) Filed: Jul. 19, 2022

Related U.S. Application Data
(60) Provisional application No. 63/223,629, filed on Jul. 20, 2021.

(51) Int. Cl. A61B 5/00; A61B 5/293; A61B 5/372; A61M 27/00

(52) U.S. Cl. CPC A61B 5/6852; A61B 5/293; A61B 5/372; A61B 5/7275; A61B 2562/0209; A61M 27/006

(57) ABSTRACT

Treatment and monitoring of diseases associated with elevated intracranial pressure (ICP) or hydrocephalus, such as traumatic brain injury (TBI), and other conditions for which an externalized ventricular drain (EVD) or ventriculoperitoneal (VP) shunt may be used includes monitoring electrical signals from an electrode positioned in a brain of the subject while the subject is being treated for the disease. The treatment may include draining cerebral spinal fluid with a drainage catheter. The drainage catheter may comprise the electrode for monitoring the electrical signals.

### RELATED APPLICATION

This application claims priority to U.S. Provisional Application No. 63/223,629 filed Jul. 20, 2021, entitled TREATMENT AND MONITORING OF DISEASES ASSOCIATED WITH ELEVATED INTRACRANIAL PRESSURE OR HYDROCEPHALUS which is incorporated herein by reference in its entirety to the extent it does not conflict with the disclosure presented herein.

### FIELD

The present disclosure relates to, among other things, treatment and monitoring of diseases associated with elevated intracranial pressure (ICP) or hydrocephalus, such as traumatic brain injury (TBI), and other conditions for which an externalized ventricular drain (EVD) or ventriculoperitoneal (VP) shunt may be used.

### INTRODUCTION

Traumatic brain injury (TBI) remains a leading cause of disability and death in children and adults in their most productive years. It is also a leading cause of morbidity and mortality in the military. Every year an estimated 1.6 million head injuries occur in the US. TBI affects three out of every 1,000 Americans annually, accounting for as many as 60,000 deaths and an estimated 70,000 to 90,000 individuals with chronic neurological disabilities. The economic consequences of these injuries are enormous in terms of lost productivity and medical care costs. The direct and indirect costs of TBI in the US are estimated to be $48.3 billion annually. Survivor costs account for $31.7 billion and fatal brain injuries amount to another $16.6 billion. While the last two decades of research has resulted in a greater understanding of the physiological and cellular events leading to secondary neuronal injury, and methods to characterize changes in cerebral blood flow and intracranial pressure have modestly improved, there remains a profound lack of brain functional measurement tools starting at the time of acute injury through rehabilitation which provide objective metrics to guide treatment decisions in patients with severe TBI.

Management of severe TBI and associated elevated intracranial pressure (ICP) typically necessitates placement of an externalized ventricular drain (EVD). This implant accesses the ventricle via a flexible tube and drains cerebral spinal fluid (CSF) to lower ICP. This procedure, common in neurosurgical practice, is a cornerstone of evidence-based care for patients presenting with severe traumatic closed head injury. Each year, approximately 23,000 EVDs are placed in the US and 4,000 (or 17%) are placed for presumed severe closed head injury.

Patients with EVDs typically go through a two-week period of intensive care during which the patient is often intubated and aggressive multisystem care is focused on preserving life and brain function. These patients typically remain in the hospital for another two or so while caregivers attempt to find placement in a long-term or rehabilitation facility.

Post-traumatic hydrocephalus is a poorly studied frequent sequalae of TBI. Management of patients with severe TBI and associated ventricular enlargement frequently necessitates placement of a VP shunt (VPS). This fully subcutaneous implant accesses the ventricle via a flexible tube and across a valve system drains CSF to the peritoneal catheter. This procedure, common in neurosurgical practice, is a cornerstone of evidence-based care for patients presenting with severe TBI. Each year, thousands of VSPs are placed in the US for a variety of indications. Patients with VPSs either are briefly admitted as an outpatient from a rehab or outpatient facility or are on their way to such a facility from being an inpatient post injury for another two weeks while caregivers attempt to find placement in a long-term or rehabilitation facility.

Challenging behavioral presentations are nearly universal for both severe TBI and post-traumatic hydrocephalus, as is use of psychotropic medications to manage them. A common course is for patients to move from an inpatient setting into at least a year of nursing care or rehabilitation, depending on their clinical course, level of consciousness, cognition, language, and behavioral disturbance. Despite the obvious criticality of the evolving brain injury, physicians lack objective data regarding brain physiology and function, relying instead on clinical observation to inform treatment decisions. After the acute injury through the first year of recovery, the clinical challenges evolve, starting when it occurs with elevated ICP or structural brain and skull damage, extending to sleep disturbance in the ICU, additional behavioral and sleep challenges in the post-extubation hospitalization period, and later switch to the cognitive, impulse control, and behavioral challenges that interfere with occupational, speech, and physical therapy that are the key approaches to optimizing brain and functional outcomes over the first year.

To date, while EVDs and VPs are surgically implanted in thousands of patients with TBI or hydrocephalus post TBI, they have been employed and designed to date solely to lessen ICP. Current EVD and VP devices provide no information about brain physiology and function despite the great need for objective biomarkers of evolving brain states and despite the fact that EVD devices are already being located physically implanted in patients' brains.

### SUMMARY

The present disclosure relates to, among other things, treating and monitoring of TBI. In some embodiments, the present disclosure describes a pathway to understand and treat TBI that includes ventricular drainage and monitoring of electrical brain signals from patients suffering from TBI.

Monitoring of electrical brain signals may include obtaining high quality intracranial brain electroencephalography (iEEG) data from TBI patients. The data may be used to better understand TBI. The data may be used to monitor a state of the disease (such as severity), a state of the patient, or the like. The data may be used to direct, enhance, or direct and enhance TBI therapy. The TBI therapy may include one or more of cognitive therapy, psychological therapy, pharmacological therapy, and ventricular drainage.

In some embodiments, CSF is drained through the use of a drainage catheter having an opening placed in a cerebral ventricle to an opening located outside of the CSF space, such as external to the patient or within a suitable drainage location such as the peritoneal cavity. CSF drainage may reduce ICP.

In some embodiments, the CSF drainage catheter contains one or more electrodes for recording signals from the patient's brain. One or more electrodes of the drainage catheter may be configured to be positioned within or close to white matter of the brain, grey matter of the brain, or white matter and grey matter of the brain. In addition or alternatively, a medical lead separate from a drainage catheter may include one or more electrodes configured to be positioned within or close to white matter of the brain, grey matter of the brain, or white matter and grey matter of the brain. Preferably, the medical lead is configured to be coupled to, or is coupled to, a drainage catheter so that implantation of the drainage catheter results in implantation of the lead, if the drainage catheter is implanted in the brain. That is, it is preferable that only one surgical procedure is performed to place the lead and the drainage catheter. The electrodes may record electrical signals from within the brain.

By placing the electrodes within or in proximity to brain tissue, such as white matter or grey matter, less “noisy” signals may be obtained than with scalp-based EEG recordings. Such higher quality signals may facilitate interpretation of data recorded by the electrodes.

Data recorded by the electrodes may be used to monitor disease or therapy progression, as well as monitor a current brain state or predict a future brain state. Electrical biomarkers may be identified. Such objective biomarkers of disturbed brain function in TBI can provide both targets for therapeutic interventions and valid metrics by which to evaluate the impact of modifications and tailoring of current available treatment strategies. This is especially important because the neuropsychiatric disturbances in TBI frequently compromise the reliability and validity of self-report. Treatment strategies guided by objective biomarkers may result in superior outcomes and shorter treatment periods in intensive care units, hospital, and rehabilitation settings.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a schematic sectional view illustrating an embodiment of an access port and a catheter having electrodes implanted in a subject.

FIG. 2 is a schematic perspective view of an embodiment of a catheter having electrodes.

FIG. 3 is a schematic view of an embodiment of implantable infusion device, signal apparatus, catheter with electrodes, and external apparatus.

FIG. 4 is a schematic view of an embodiment of an external apparatus being worn around an ear of a subject.

FIG. 5 is a flow diagram illustrating an embodiment of a method for developing a PTSD Deep Neural Network (DNN).

FIG. 6 is a flow diagram illustrating an embodiment of a method for applying a PTSD DNN to monitor or treat PTSD.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

Like numbers used in the figures refer to like components and steps. However, it will be understood that the use of a number to refer to a component in a given figure is not intended to limit the component in another figure labeled with the same number. In addition, the use of different numbers to refer to components in different figures is not intended to indicate that the different numbered components cannot be the same or similar to other numbered components.

### DETAILED DESCRIPTION

The present disclosure relates to, among other things, methods, devices, and systems for treating, monitoring, or treating and monitoring a disease associated with elevated ICP or hydrocephalus, such as TBI.

Monitoring of a disease associated with elevated ICP or hydrocephalus preferably includes obtaining high quality electrical signals relating to brain activity obtained by one or more electrodes positioned in the white or grey matter of a patient suffering from the disease associated with elevated ICP or hydrocephalus, such as TBI. White matter of the brain is composed mainly of long-range myelinated axons, and as such serves as a preferred target for monitoring general electrical activity of the brain. Preferably, the electrodes in the white matter capture electrical activity associated with general brain state rather than merely capturing signals localized to small regions of the brain. The high-quality brain activity signals recorded by electrodes in white matter may facilitate interpretation processing and analysis of the signals, which may be used for any suitable reason. Grey matter of the brain is composed mainly of neuronal cell bodies. The electrodes in grey matter primarily capture local field potentials, or electrical activity associated with local regions of brain. Recording activity within confined brain regions, or interactions between two or more grey matter regions may facilitate interpretation, processing and analysis of the signals, which may be used for any suitable reason.

By collecting high quality data derived from within the brain (iEEG) and Artificial Intelligence (AI) techniques, an effective disease (e.g., TBI) deep neural network (DNN) may be obtained. The disease (e.g., TBI) DNN may be used for patient management. Additionally, the disease (e.g., TBI) DNN may support the construction of a non-invasive symptom management and diagnostic tool for non-implanted patients that may be suffering from or at risk of a disease associated with elevated ICP or hydrocephalus, such as TBI.

DNNs are a sub-field of machine learning which leverage a composition of many nonlinear functions to map input data into a new desired output domain. The parameters of these nonlinear functions are not directly designed by humans, but instead learned from vast quantities of data. This allows the continual learning and improvement of a DNNs performance through the collection of more high-quality data. DNNs have found widespread success across numerous domains that often match or surpass human performance on specific tasks. AI may form the basis for iEEG analysis emphasizing those deep learning strategies initially shown most helpful for waking EEG classification for emotions, motor activity, cognitive activity, seizure detection and sleep scoring including convolutional neural networks and recurrent neural networks; utilizing both supervised and unsupervised training approaches. The course of sleep disturbances, a primary symptom of PTSD, may include ongoing analysis with local field potentials from deep brain electrodes subject to ongoing analysis by modern machine learning techniques as well as classical methods such as support vector machine and decision tree methods.

Suitable AI methods for establishing DNNs are described in U.S. Provisional Patent Application No. 63/054,522, entitled MONITORING AND TREATMENT BASED ON CONTINUOUS INTRACRANIAL EEG ACTIVITY, filed on Jul. 29, 2020, and naming Cerebral Therapeutics, Inc. as an Applicant, which is incorporated herein by reference in its entirety to the extent that it does not conflict with the disclosure presented herein.

The disease (e.g., TBI) DNN, initially trained on data derived from implanted iEEG, may learn the important feature representations associated with disease (e.g., TBI). These learned feature representations may be adapted for use on a noisier, lower quality, non-invasive data from brain electrical recordings, such as recordings from electrodes place on a subject's scalp. Thus, monitoring and treatment of a disease associated with elevated ICP or hydrocephalus (e.g., TBI) using a disease (e.g., TBI) DNN developed from high quality iEEG recordings may prove beneficial to larger patient populations through application of the disease (e.g., TBI) DNN to non-invasive techniques.

An example of a device or system that may be used to treat and monitor a disease associated with elevated ICP or hydrocephalus (e.g., TBI) will now be described. It will be understood that other devices and systems may be employed in accordance with the principles described herein.

Information from the recorded electrical signals or disease (e.g., TBI) DNNs may be used to understand a current brain state or to predict a future brain state of a patient suffering from a disease associated with elevated ICP or hydrocephalus. Preferably, the electrical signals or disease DNNs may be employed to modify and improve treatment of the disease. Treatment of a disease associated with elevated ICP or hydrocephalus, such as TBI, may include one or more of cognitive therapy, psychological therapy, pharmacological therapy, physical therapy, and CSF drainage.

A CSF drainage catheter (e.g., an EVD or a ventriculoperitoneal shunt) may include one or more electrodes configured to be positioned within or close to white matter of the brain, grey matter of the brain, or white matter and grey matter of the brain. In addition or alternatively, a medical lead separate from a drainage catheter may include one or more electrodes configured to be positioned within or close to white matter of the brain, grey matter of the brain, or white matter and grey matter of the brain. Preferably, the medical lead is configured to be coupled to, or is coupled to, a CSF drainage catheter so that implantation of the catheter results in implantation of the lead, if the catheter is implanted in the brain. That is, it is preferable that only one surgical procedure is performed to place the lead and the drainage catheter. The electrodes may record electrical signals from within the brain.

A drainage catheter may be modified or adapted to include one or more electrodes, which are located on the catheter such that they are configured to be positioned in or near white or grey matter of the brain following implantation of the catheter. The electrodes preferably record electrical signals from the white matter.

A catheter as described herein includes a proximal end, a distal end portion having a distal tip, at least one lumen extending from the proximal end to the distal end portion. The catheter may comprise one or more electrodes. The one or more electrodes are positioned on the catheter a distance from a distal tip such that the one or more electrodes would be placed in contact with white or grey matter of the brain if the distal tip of the catheter were at a suitable target location of the brain for CSF drainage.

CSF exits the foramen of Magendie and Luschka to flow around the brainstem and cerebellum. CSF flows within the subarachnoid space. CSF is produced in the ventricular system of the brain and communicates freely with the subarachnoid space via the foramen of Magendie and Luschka. The distal tip of the catheter may be placed anywhere that the CSF is accessible. For example, the distal tip of the catheter may be placed in communication with the cisterna magna or a cerebral ventricle. Preferably, the distal tip is placed in a cerebral ventricle. Preferably, the cerebral ventricle is a lateral cerebral ventricle.

Additional details regarding suitable catheters including recording electrodes for recording electrical signals from the brain are described in U.S. Provisional Patent Application No. 63/053,864, entitled FLUID CATHETER DEVICE FOR RECORDING BRAIN STATE, filled on Jul. 20, 2020, and naming Cerebral Therapeutics, Inc. as an Applicant, which provisional patent application is hereby incorporated by reference in its entirety to the extent that it does not conflict with the disclosure presented herein.

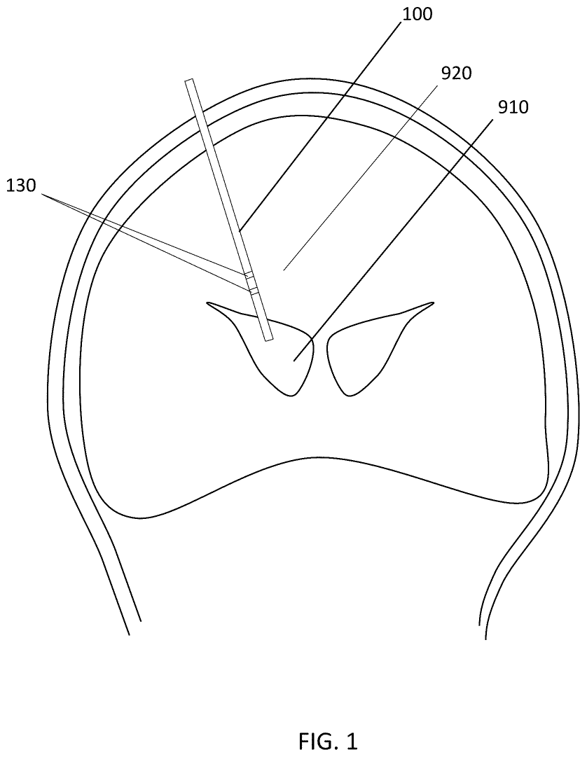

Referring now to FIG. 1, a catheter 100 is shown implanted in a patient. The catheter 100 extends from external to a patient to a lateral ventricle 910. The catheter 100 has a lumen for draining CSF from the lateral ventricle 910. Electrodes 130 of the catheter 100 are positioned in the white matter 920 or grey matter of the brain.

While the catheter 100 depicted in FIG. 1 is positioned and configured to drain CSF external to a patient, the CSF may be drained internal to a patient, such as into an intraperitoneal space. Preferably, the catheter 100 is a long-tunneled EVD. A long-tunneled EVD may be an EVD that exits the skin of the patient at a distance of 10 cm or more from a location of a burr hole in the skull in which the catheter is inserted. The catheter 100 may exit the skin at a location that is 20 cm or more from the burr hole, 30 cm or more from the burr hole, 40 cm or more from the burr hole, or 50 cm or more from the burr hole. For example, a long-tunneled EVD may be externalized at an abdominal level, at the level of an anterior chest wall, or the like. See, for example, C. D. E. Collins et al., Childs Nerv. Syst. (2014) 30:1671-1678 (DOI 10.1007/s00381-014-2523-3).

The catheter 100 may have any suitable length. For example, the catheter 100 may have a length of 10 cm or more, 20 cm or more, 30 cm or more, 40 cm or more, or 50 cm or more.

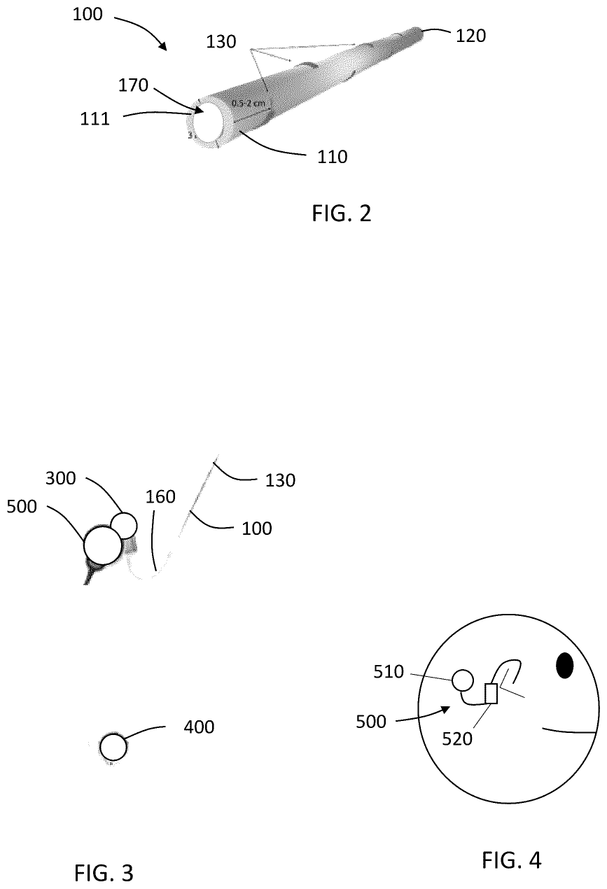

As shown in FIG. 2, a catheter 100 has a proximal end 120, a distal tip 111 at a distal end region 110, and a lumen 172 extending from the proximal end 120 to the distal tip 111. In some embodiments (not shown) the lumen 172 does not extend to the distal tip 111, but rather the distal end region 110 comprises openings (not shown) in communication with the lumen 172, 174 through which fluid may flow. The depicted catheter 100 includes two lumens 172, 174, separated by a wall 180 that extends the length of the catheter 100. However, the catheter 100 may have any suitable number of lumens.

The catheter 100 has electrodes 130 positioned along a length of the catheter beginning at a distance from the distal tip 111, such as about 0.5 centimeters to about 2 centimeters. The electrodes 130 may span a distance along the length of the catheter 100, such as about 3 centimeters to about 5 centimeters. Conductors (not shown) are operably coupled to the electrodes 130 and may serve to electrically couple the electrodes 130 to signal apparatus.

Alternatively, a medical lead, separate from the catheter, and having recording electrodes may be employed.

The devices, systems, or devices and systems describe herein may include signal apparatus electrically coupled to the electrodes. For example, the signal apparatus may be electrically coupled to the electrodes via an electrical interconnect. The signal apparatus may process, transmit, or process and transmit data regarding the signals recorded by the electrodes. The signal apparatus may comprise any suitable components, such as components configured to one or more of: amplify, digitize, filter, and transmit data regarding the electrical signals recorded by the electrodes. For example, the signal apparatus may comprise one or more of: an amplifier, an analog to digital converter, a band pass filter, an antenna, and a transmission coil.

The signal apparatus may be configured to sample the signals from the electrodes at any suitable frequency. For example, the signal apparatus may be configured to sample the signals at a frequency of about 100 hertz or greater, such as 1,000 Hertz or greater. Preferably, the signal apparatus is configured to sample the signals at a frequency of about 10,000 hertz or greater. The signal apparatus may process the signal at any suitable bit depth, 4 bits, 8 bits, 16

The signal apparatus may be implanted in the patient at any suitable location. The signal apparatus may comprise a power source or may be wirelessly powered. If the signal apparatus is wirelessly powered, the signal apparatus preferably includes an inductive coil, solenoid, or other suitable components to be wirelessly powered by an external apparatus and to transmit data regarding the signals recorded by the electrodes to the external apparatus. The signal apparatus is preferably implanted at a location where it may inductively couple with the apparatus external. For example, the signal apparatus may be positioned under the scalp of the subject near an ear of the subject. Such positioning may allow the external apparatus to be comfortably worn on or around the ear of the subject to provide suitable inductive coupling to power the signal apparatus and to wirelessly transmit data regarding the signals recorded by the electrodes from the signal apparatus to the external apparatus. The external device may then transfer the data to the cloud or to another device, such as a smart phone, a personal computer, or the like, which may then transfer the data to a server, or the like.

For example and referring to FIGS. 3 and 4, a catheter 100 comprising electrodes 130 for recording electrical signals in the brain may be electrically coupled to signal apparatus 300 configured to process, transmit, or process and transmit data regarding the electrical signals recorded by the electrodes 130. External apparatus 500 may be positioned relative to signal apparatus 300 such that the data may be transmitted wirelessly from the signal apparatus 300 to the external apparatus 500. The external apparatus 500 may be configured to wirelessly power the signal apparatus 300 in some embodiments.

For example and with reference to FIG. 3, the signal apparatus 300, which is coupled to electrodes 130 by conductors that run through cable 160 and through or along a portion of the catheter 100, is implanted under the skin such that external apparatus 500 may wirelessly power and receive data from signal apparatus 300. For example and with reference to FIG. 4, the signal apparatus may be implanted under the subjects scalp near the ear. External apparatus 500 comprises an inductive coupling component 510 that may be positioned over the signal apparatus and comprises a processing component 520 operably coupled to the inductive coupling component 510. The processing component 520 may include, among other things, a rechargeable battery and a processor. The external apparatus 500 may transmit data received from signal apparatus 300, or a processed version thereof, to suitable secondary device, such as a smartphone, personal computer, tablet, modem, or the like through any suitable platform, such as low power Bluetooth. The secondary device may transmit data to the internet, where the data may be stored or retrieved by other computing devices as appropriate.

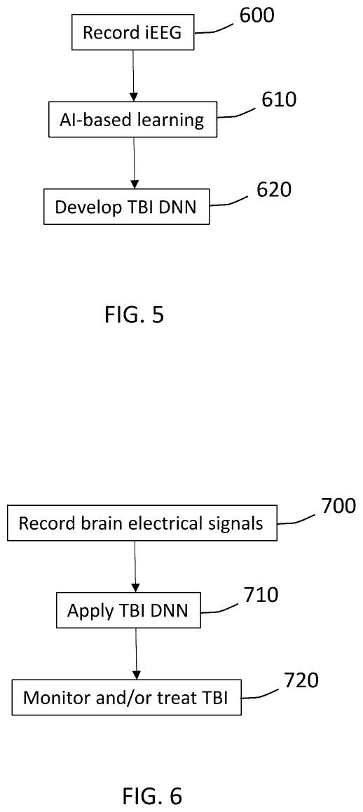

Referring now to FIG. 5, a flow diagram illustrating an embodiment of a method for developing a disease DNN (such as a TBI DNN) is shown. The method may include collecting brain electrical data from one or more patients suffering a disease associated with hydrocephalus or elevated ICP (e.g., TBI) by iEEG recordings (600). AI-based learning approaches (610) may be used to identify feature representations from the high-quality data to develop a disease DNN (e.g., TBI-DNN) (620). The AI-based learning approaches may be aided by manual intervention. The AI-based learning approaches may be semi-automated or fully automated. As indicated above, suitable AI methods for establishing DNNs are described in U.S. Provisional Patent Application No. 63/054,522, entitled MONITORING AND TREATMENT BASED ON CONTINUOUS INTRACRANIAL EEG ACTIVITY, filed on Jul. 21, 2020, and naming Cerebral Therapeutics, Inc. as an Applicant, which is incorporated herein by reference in its entirety to the extent that it does not conflict with the disclosure presented herein.

Referring now to FIG. 6, a flow diagram illustrating an embodiment of a method for applying a disease DNN (e.g., TBI) for monitoring or treating a disease associated with elevated ICP or hydrocephalus, such as TBI, is shown. The method includes recording electrical brain signals (700), which may include iEEG recordings or may include lower quality, more noisy recordings, such as recordings obtained from electrodes placed on a subject's scalp. The disease DNN (e.g., TBI-DNN) may be applied (710) to the recorded brain electrical signals to monitor, treat, or monitor and treat the disease, such as TBI (720).

While of skill in the art will readily understand methods that may be employed to the devices and systems described herein. A brief description of some of the methods contemplated herein are described below.

In some embodiments, a method comprises recording an electrical signal from white or grey matter of a brain of a subject suffering from TBI and determining a brain state of the subject or predicting a future brain state of the subject based on the recorded electrical signal.

Recording the electrical signal from the white or grey matter of the brain of the subject may comprise recording the signal from one or more recording electrodes positioned in the white or grey matter. The recording electrodes may be on a catheter configured to drain CSF from a brain of the subject or may be separate from the catheter. The catheter may be positioned to drain CSF from a cerebral ventricle of the subject.

A method may comprise applying an electrical signal to the white or grey matter. Recording the electrical signal from the white or grey matter of the brain of the subject may comprise recording an electrical response evoked by the applied electrical signal. Monitoring an evoked response may advantageously reduce relatively high intrinsic noise that may be present when passively monitoring, which noise may occlude a desired signal. Active monitoring (i.e., recording an evoked response) may improve the signal to noise ratio relative to passive monitoring. The electrical signal may be applied at regular intervals and the changes in evoked response recorded.

The method may comprise transmitting data regarding the recorded electrical signal to apparatus external to the subject. The method may comprise determining a brain state of the subject or predicting a future brain state of the subject based on the recorded electrical signal.

In some embodiments, a method includes monitoring the safety or effectiveness of the CSF drainage therapy or other therapy for treating a patient suffering from TBI. Because the subject has an electrode positioned to record electrical signals from white or grey matter implanted in their brain, the recorded electrical signals may be used to monitor any therapy that the subject is undergoing.

While this disclosure is particularly directed to treatment and monitoring of TBI, it will be understood that principles described herein may be applied to other diseases for which the use of a CSF drainage catheter may be applicable, such as a disease associated with increased intracranial pressure. The principles described herein may have an impact in early identification and treatment before disorders become chronic and disabling.

Examples of diseases which may be treated by ventriculostomy include those discussed in B. P. Rosenbaum et al., Journal of Clinical Neuroscience 21 (2014) 623-632 (https://doi.org/10.1016/J.JOCN.2013.09.001), such as subarachnoid hemorrhage, intracerebral hemorrhage, subdural hemorrhage, extradural hemorrhage, obstructive hydrocephalus, nervous system cancer (such as secondary malignant neoplasm of the brain, spinal cord, or other parts of the nervous system; malignant neoplasm of the cerebellum nos; malignant lymphoma of an unspecified site, extranodal site, or solid organ site; and the like), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (such as due to nervous system device, implant or graft), mechanical complication of a nervous system device, implant or graft). Other diseases that may be treated by ventriculostomy include TBI, congenital hydrocephalus, aneurysms, and the like. Diseases treatable by ventriculostomy, e.g., with an EVD, may also be treated with a CSF drainage shunt, such as a ventriculoperitoneal (VP) shunt.

In some embodiments, a patient suffering from a disease associated with elevated ICP or hydrocephalus may be treated with an EVD and a VP shunt. The treatment with the EVD and VP shunt may be concurrent or may occur at different times. For example, the patient may first be treated with an EVD and then be treated with a VP shunt.

All scientific and technical terms used herein have meanings commonly used in the art unless otherwise specified. The definitions provided herein are to facilitate understanding of certain terms used frequently herein and are not meant to limit the scope of the present disclosure.

As used herein, “treat,” “treatment,” or the like mean to reduce or alleviate one or more symptom or to slow the progression of the disease being treated.

As used herein, singular forms “a,” “an” and “the” include plural referents unless the context clearly dictates otherwise. As used in this specification and the appended claims, the term “or” is generally employed in its sense including “and/or” unless the content clearly dictates otherwise. The term “and/or” means one or all the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the inventive technology.

Any direction referred to herein, such as “top,” “bottom,” “side,” “upper,” “lower,” and other directions or orientations are described herein for clarity and brevity but are not intended to be limiting of an actual device or system. Devices and systems described herein may be used in a number of directions and orientations.

As used herein, “providing” an article, device, or system means manufacturing the article, device, or system, assembling the article, device, or system, purchasing the article, device, or system, or otherwise obtaining the article, device, or system.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The headings used herein are for organizational purposes only and are not meant to be used to limit the scope of the description. As used throughout this application, the word “may” is used in a permissive sense (i.e., meaning having the potential to), rather than the mandatory sense (i.e., meaning must).

The words “include,” “including,” and “includes” indicate open-ended relationships and therefore mean including, but not limited to. Similarly, the words “have,” “having,” and “has” also indicated open-ended relationships, and thus mean having, but not limited to. Similarly, the terms “comprise” and “comprising” indicate open-ended relationships, and thus mean comprising, but not limited to. The terms “consisting essentially of” and “consisting of” are subsumed within the term “comprising.” For example, a catheter comprising tubing may be a catheter consisting of tubing. The term “consisting essentially of” means a recited list of one or more items belonging to an article, kit, system, or method and other non-listed items that do not materially affect the properties of the article, kit, system, or method.

The terms “first,” “second,” “third,” and so forth as used herein are used as labels for nouns that they precede, and do not imply any type of ordering (e.g., spatial, temporal, logical, etc.) unless such an ordering is otherwise explicitly indicated. For example, a “second” feature does not require that a “first” feature be implemented prior to the “second” feature, unless otherwise specified.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task (e.g., a catheter connector may be configured to place a lumen of a catheter in fluid communication with a fluid path, even when the catheter is not connected to the catheter connector).

Various components may be described as performing a task or tasks, for convenience in the description. Such descriptions should be interpreted as including the phrase “configured to.” Reciting a component that is configured to perform one or more tasks is expressly intended not to invoke 35 U.S.C. § 112 paragraph (f), interpretation for that component.

### EXAMPLES

### Example 1

### Continuous Implanted High Quality EEG Monitoring (and Development of EEG Deep Neural Networks for Improving TBI Rehabilitation) During and After Vventriculostomy for Individuals with Severe TBI

Collection of high quality, continuous iEEG data may occur during clinician management of TBI. A first step may be focus on epileptiform and sleep stage detection (high risk and frequent complications for these patients). A second may be to expand the data scope applying labeling strategies with the continuous iEEG during the course of their recovery to identify patterns of brain electrical activity that reflect abnormal brain states and/or covary with behavioral disturbance.

In initial phases after injury, detection of undiagnosed epileptiform conditions and sleep disorders may be prioritized using conventional techniques combined with AI methods. As is demonstrated in an epilepsy iEEG pilot data section, advanced AI-DNN methods may subsequently be applied to drive the identification of potential biomarkers, emphasizing and building on application of those deep learning strategies shown most helpful for EEG classification of sleep stages and epileptiform activity (data not shown). This approach may be expanded from sleep and epileptiform activity to iEEG-DNNs linked to specific disturbances in emotion, motor activity, or cognition. The characterization of sleep disturbances, which may evolve with closed head injury, may include longitudinal analysis of local field potentials from deep brain electrodes using modern machine learning techniques, as well as classical methods, such as support vector machine and decision tree methods. The availability of recordings from both cortical and deep white matter regions may facilitate control for brain state non-homogeneity during sleep states.

During the subsequent phases of recovery, in addition to providing iEEG objective biomarkers of sleep and epileptiform disturbance, it is postulated that the iEEG-DNN approach may provide objective biomarkers useful in the diagnosis and ultimately the management of behavioral disturbances, such as agitation, inattention, or disorientation. Behavioral disturbance in the TBI patient are often incompletely or poorly controlled with psychotropics. Since the iEEG can be continuously collected and because the iEEG implant can remain in the patient even after the EVD has been removed, the TBI DNN strategy can then be expanded over the longer course of recovery to explore the potential of biomarker identification for a variety of neuropsychiatric complications, including sleep disturbances, agitation, PTSD, suicidal and impulsive behaviors.

Detecting EEG sleep and epileptiform activity patterns using an extremely more capable iEEG collecting implant may enable more accurate and clinically meaningful assessment of change in these domains. Given our knowledge of sleep architecture and the nature of epileptiform activity, disturbances in these domains are likely analyzable based on iEEG analysis alone. However, the development of TBI iEEG DNNs may be important for many brain dysfunctions, such as agitation, suicidality, or attentional dysfunction which may require linkage between the brain data and behavioral manifestations. Tagging strategies may be used for such linkage by an experienced AI data analytic and specific PTSD psychiatry team.

Cerebral Therapeutics, Inc. has previously analyzed a pilot data set from a unique epilepsy cohort with just this type of data analytic approach and was able to recognize sleep stages and epileptiform discharges comparable with the proposed modified iEEG

In Aim 1, a minimally invasive ventriculostomy catheter and iEEG monitoring device may be used in a pilot study of 20 severe TBI participants to demonstrate potential clinical utility (effective CSF drainage and quality sleep and EEG data captured as compared with scalp EEG data capture). In Aim 1, specific iEEG data signatures for epileptiform activity and sleep disturbance will be generated. The iEEG device acquisition system may record from 2× electrodes distributed over a intracranial depth of approximately 4× cm, with the signals externally digitized (250 Hz sampling rate). This arrangement may produce approximately 0.095 Gigabyte of data per day per patient (about 3 GB per month per patient). The internalized system may be compatible with routine use of ventriculostomy and externalized iEEG capture and digitalization technology may be wearable, and ergonomically and unobtrusively designed, to support cloud-based data transfer.

In Aim 2, the pilot study findings may be expanded to include preliminary application of TBI DNN strategy to the domains of agitation, cognitive impairment, and, when appropriate, PTSD or suicidal ideation. Just as innovation and advanced analytic techniques may be applied to the iEEG data, state-of-the-art methods may be applied when “labelling” contemporaneous behavioral events. The behavior collection strategies may include analysis of video recordings, nursing and caregiver daily reports, medications and their administration times, continuous recording of ongoing physiology (e.g., heart rate, respiration, etc.) and, when feasible, self-report of subjective states and objective task performance.

After successfully addressing Aims 1-2, the follow-on stages of development of the may proceed, progressing towards commercialization and full regulatory (e.g., FDA) clearance.

Specifically, this may mean conducting an appropriately powered and controlled clinical trial demonstrating the clinical utility and safety of the technology and iEEG analyses. The design and implementation of a current Phase 2b epilepsy trial conducted by Cerebral Therapeutics, Inc. may be helpful to model an approach for the proposed TBI patient investigation.

A primary significance of the proposed research resides in the collection and utilization of unique brain data and novel analytic strategies to develop biomarkers in a population with severe TBI. There are four major areas of significance in this realm:
1) The collection of high-quality brain data, built upon solid experience with acquisition and analysis of intraventricular sleep and epilepsy continuous iEEG, has never been routinely obtained in patients with severe brain injury, either acutely or over the long-term over the course of their brain injury and recovery. Acquiring these key data provides an unparalleled opportunity for the field: unique high-quality, in the brain, objective data by which to investigate the optimal delivery of rehabilitation interventions and prescriptions (i.e., frequency, intensity, timing, and type), as well as to investigate the comparative effectiveness of standard of care and novel intervention strategies. The functional brain data gap is not unique among brain diseases to severe TBI. However, what is unparalleled is the opportunity to access and analyze these unique brain data with minimal or no added risk and tremendous possibility of gain for patients and the field. Thousands of such patients have EVDs implanted to reduce ICP. Yet, these EVDs currently provide no information about brain physiology or function.
2) During the acute and first weeks of treatment, the continuous iEEG data may be used to monitor for epileptiform activity and sleep disruption. Sleep disturbance and epileptiform activity following TBI are well known to disrupt recovery, impacting on symptom manifestation and behavioral changes, and the management of sleep disturbance and epileptiform activity can be improved upon in many severe TBI patients. Once EEG signatures described in (1) above have been obtained, AI DNN analytic strategies may be utilized and compared with surface EEG and other physiological measures (e.g., activity counts, heart rate and variability, skin conductance measures and others) to initially test sensitivity and specificity in detecting and predicting sleep disturbances and indicators of seizure activity. Based on the much higher quality data, it is expected that the biomarkers identified with ciEEG and AI DNN detection may demonstrate stronger sensitivity and specificity than scalp EEG.
3) After the first 4-6 weeks phase, the iEEG data may continue to be collected, along with additional monitoring for attentional challenges and anxiety symptoms which may trigger oral medication intervention to help patients improve their post-injury course. Similar iEEG and DNN AI techniques and strategies may be applied as in Aim 1, but at this point with tagging to specific behavioral states (e.g., agitation) or subjective states (PTSD, suicidality).
4) Objective biomarkers in TBI of abnormal brain states and behavioral disturbances have never been previously identified and could radically alter clinical management by providing objective and outcome relevant endpoints. This data-driven, empirical strategy represents a major scientific advance that could be applied to other forms of behavioral disturbance and their accompanying altered brain states, such as mood disorders and substance misuse, which also represent major problems for both military and civilian populations.

### Example 2

### Continuous Implanted High Quality EEG Monitoring (and Development of EEG Deep Neural Networks for Improving TBI Rehabilitation) During and After Concurrent with VP Shunting for ICP Management for Individuals with Hydrocephalus Post Severe TBI

One goal of this prophetic study is to develop objective tools whose application will shorten rehabilitation time and improve patient quality of life (QOL) after severe TBI. While VPSs are surgically implanted in thousands of patients, including a large number with TBI, to date they have been employed and designed to chronically drain CSF but have not been employed to derive information about short or long term brain function despite the significant need for objective data to optimize management of severely impacted patients.

The inventors believe that leveraging new monitoring technology and high quality implantable EEG data and AI analytic approaches, which have been developed for monitoring epilepsy patients, to TBI patients receiving VPSs will yield objective behaviorally-significant biomarkers to improve care across the TBI treatment and recovery period. The identification of objective biomarkers of disturbed brain function in TBI can provide targets for therapeutic interventions and metrics by which to evaluate the impact of treatment modifications. Treatment strategies guided by objective biomarkers may result in superior outcomes and shorter treatment periods in ICU, hospital, and rehabilitation settings.

In Aim 1, a minimally invasive ventriculostomy catheter and iEEG monitoring device may be used in a pilot study of 20 severe TBI VPS patients to demonstrate clinical utility (without interfering with effective VP shunt CSF drainage) and quality iEEG data captured as compared with scalp EEG). The first generation iEEG VPS device acquisition system may record from 2 electrodes distributed over an intracranial depth of ~4 cm, with the signals externally digitized (250 Hz sampling rate). The system may be be compatible with routine VPS's use. The iEEG capture and digitalization technology may be wearable and ergonomically designed to support cloud-based data transfer. High quality, continuous iEEG data may be collected during clinician management post VP Shunt insertion in candidate TBI patients. The first step may focus on epileptiform and sleep stage detection (high risk and frequent complications for these patients). Previous research with intracranial EEG has demonstrated that the occurrence and time course of sleep and epileptiform activity with focal onset are detectable remotely in the brain as long as the EEG electrodes are intracranial, recording over time, and have good signal to noise ratio (data not shown).

In the initial phases after injury, detection of undiagnosed epileptiform conditions, cortical spreading depolarizations associated with poor head injury outcomes and sleep abnormalities may be prioritized using conventional EEG analysis techniques combined with AI iEEG methods. Advanced AI-DNN methods may be applied to drive the identification of potential biomarkers, building on application of deep learning strategies shown most helpful for EEG classification of sleep stages and epileptiform activity. This approach may be expanded to iEEG-DNNs linked to specific disturbances in emotion, motor activity, or cognition. Detecting EEG sleep and epileptiform activity patterns the iEEG implant may enable more accurate and clinically meaningful assessment of change in these domains. Due to experience and knowledge of sleep architecture and the nature of epileptiform activity, disturbances in these domains are likely analyzable based on iEEG analysis alone. However, the development of TBI iEEG DNNs may be important for many brain dysfunctions such as agitation, suicidality, or attentional dysfunction which will require linkage between the brain data and behavioral manifestations. Tagging strategies may be used for such linkage by an experienced AI data analytic and specific PTSD EEG knowledgeable psychiatry team.

Since the iEEG can be continuously collected and because the iEEG implant can remain in the patient even after the EVD has been removed, during the subsequent phases of recovery, the TBI DNN strategy may then be expanded over the longer course of recovery to explore the potential of biomarker identification for a variety of neuropsychiatric complications.

In Aim 2, we may expand the pilot study findings to include preliminary application of the TBI DNN strategy to the domains of agitation, cognitive impairment, and, when appropriate, psychiatric symptomatology including PTSD or suicidal ideation. State-of-the-art methods may be applied when labelling contemporaneous behavioral events. The behavior collection strategies may include analysis of video recordings, nursing and caregiver daily reports, medications and their administration times, continuous recording of ongoing physiology and, when feasible, self-report of subjective states and objective task performance. Behavioral disturbances in the TBI patient are often incompletely or poorly controlled with psychotropics. Insight into how the ongoing psychotropics affect the iEEG signatures may be key to improving rehabilitative care.

Significance of the proposed research, at least in part, resides in the collection and utilization of unique high quality brain data and analytic strategies to develop meaningful biomarkers in a population with severe TBI lacking useful objective physiology brain measures. There are four major areas of significance within this domain:
1) The collection of high-quality brain data, built upon solid experience with acquisition and analysis of intraventricular sleep and epilepsy continuous iEEG, has never been routinely obtained in patients with severe brain injury, either acutely or over the long-term course of their brain injury and recovery. Acquiring these key data provides an unparalleled opportunity for the field: unique high-quality, in the brain, objective data by which to investigate the optimal delivery of rehabilitation interventions and prescriptions (i.e., frequency, intensity, timing, and type), as well as to investigate the comparative effectiveness of standard of care and novel intervention strategies. The functional brain data gap is not unique among brain diseases to severe TBI. However, what is unparalleled is the opportunity to access and analyze these unique brain data with minimal or no added risk and tremendous possibility of gain for patients and the field. Thousands of such patients have VPSs implanted to treat hydrocephalus. Yet, these VPSs currently provide no information about brain physiology or function.
2) During the intial weeks of monitoring post VPS insertion, the continuous iEEG data may be used to monitor for changes in epileptiform activity, spreading cortical depressions associated with severe brain damage and sleep pattern disruption. Sleep disturbance and epileptiform activity following TBI are well known to disrupt recovery, impacting on symptom manifestation and behavioral changes, and the management of sleep disturbance and epileptiform activity can be improved upon in many severe TBI patients. Once EEG signatures described in (1) above have been obtained, AI-DNN analytic strategies will be utilized and compared with surface EEG and other physiological measures (e.g., activity counts, heart rate and variability, skin conductance measures and others) to initially test sensitivity and specificity in detecting and predicting sleep disturbances and indicators of seizure activity. Based on the much higher quality data collected with the devices described herein, the biomarkers identified with iEEG and AI DNN detection may demonstrate substantially stronger sensitivity and specificity than scalp EEG.
3) As a part of Aim 2, after the first 4-6 weeks acute phase, the iEEG data may continue to be collected, along with additional monitoring focused on unlocking attentional and anxiety TBI-related dysfunction which often triggers oral medication intervention during their post-injury course. Similar iEEG and DNN-AI techniques and strategies may be applied as in Aim 1, but at this point with tagging to specific behavioral states (e.g., agitation) and/or subjective states as they may arise (PTSD, suicidality).
4) Objective biomarkers in TBI of abnormal brain states and behavioral disturbances have never been previously identified and could alter clinical management by providing objective and outcome relevant endpoints. This data-driven, empirical strategy to brain clinically significant dysfunction represents a major scientific advance that could be applied to other forms of behavioral disturbance and their accompanying altered brain states, such as mood disorders and substance misuse, which represent major problems for both military and civilian populations.

Identifying and effectively treating TBI patients for sleep disturbances may also result in the diminution of the adverse consequences and costs associated with untreated or inadequately treated TBI in a number of biological domains. Depression and substance abuse are common comorbidities with TBI, aggravated by continuation of TBI symptoms. Severe TBI may be accompanied by increases in inflammatory markers including interleukin 6, interleukin 1β, TNFα, and interferon γ levels, supporting low grade inflammation with adverse pathophysiological implications including decreased neurogenesis leading to brain volume loss, impairment in memory formation and consolidation. TBI is also accompanied by increases in REM sleep without atonia, a harbinger of REM behavior disorder with its associated increases in risk for future neurodegenerative disease, all of which may benefit from TBI iEEG biomarkers to facilitate potential targeted therapeutic drug development to change outcomes.

This approach to biomarker development and treatment guidance has the potential to enhance recovery rates and quality of life in U.S. military service members, veterans, and civilians. Further, this approach will reduce the burden of care on the Military Health System, the VA, and the civilian medical community. The development of such new strategies for identification and modulation of brain states accompanying other significant behavioral disorders such as mood disorders, anxiety disorders, problems associated with substance misuse and related issues will have wide ranging impact in terms of early identification and treatment before disorders become chronic and disabling.

It will be apparent to those skilled in the art that various modifications and variations can be made to the present inventive technology without departing from the spirit and scope of the disclosure. Since modifications, combinations, sub-combinations and variations of the disclosed embodiments incorporating the spirit and substance of the inventive technology may occur to persons skilled in the art, the inventive technology should be construed to include everything within the scope of the appended claims and their equivalents.

1. A method comprising:
selecting a subject suffering from a disease associated with elevated intracranial pressure (ICP) or hydrocephalus;
inserting a catheter into a cerebrospinal fluid (CSF)-containing space of the subject;
treating the subject for the disease, wherein treating the subject comprises draining CSF from the CSF-containing space of the subject via the catheter;
monitoring electrical signals from an electrode positioned in a brain of the subject while the subject is being treated for the disease including:
monitoring electrical signals during a catheter insertion procedure; and
monitoring electrical signals after the catheter insertion procedure,
wherein monitoring electrical signals after the catheter insertion procedure includes monitoring electrical signals during an initial recovery phase and monitoring electrical signals during a subsequent recovery phase;
generating an artificial intelligence (AI) model based on the electrical signals; and
modifying the treatment of the subject based on the AI model.

2. The method of claim 1, wherein selecting a subject suffering from the disease comprises selecting a subject suffering from traumatic brain injury (TBI) or hydrocephalus post TBI.

3. The method of claim 1, wherein selecting a subject suffering from the disease comprises selecting a subject suffering from subarachnoid hemorrhage, intracerebral hemorrhage, subdural hemorrhage, extradural hemorrhage, obstructive hydrocephalus, nervous system cancer (which may include one or more of secondary malignant neoplasm of the brain, spinal cord, or other parts of the nervous system; malignant neoplasm of the cerebellum nos; or malignant lymphoma of an unspecified site, extranodal site, or solid organ site), cerebral artery occlusion, unspecified cerebral infarction, closed fracture of the base of the skull (which may be associated with one or more of subarachnoid hemorrhage, subdural hemorrhage, extradural hemorrhage, and loss of consciousness), infection and inflammatory reaction (which may be due to nervous system device, implant or graft, mechanical complication of a nervous system device, implant or graft), aneurysm, or congenital hydrocephalus.

4. The method of claim 1, wherein the catheter comprises a proximal end, a distal end portion, and a lumen extending from the proximal end to the distal end portion, wherein the distal end portion is placed in the CSF-containing space, and wherein a portion of the catheter extends through a burr hole in a skull of the subject.

5. The method of claim 4, wherein the CSF-containing space is a cerebral ventricle.

6. The method of claim 5, wherein the cerebral ventricle is a lateral ventricle.

7. The method of claim 4, wherein the CSF is drained to a peritoneal cavity of the subject.

8. The method of claim 4, wherein the CSF is drained external to the subject.

9. The method of claim 8, comprising externalizing the proximal end of the catheter through a skin location of the subject.

10. The method of claim 9, wherein the skin location is 10 cm or more from the burr hole.

11. The method of claim 9, wherein the skin location is 20 cm or more from the burr hole.

12. The method of claim 10, wherein the skin location is 30 cm or more from the burr hole.

13. The method of claim 10, wherein the skin location is 40 cm or more from the burr hole.

14. The method of claim 10, wherein the skin location is 50 cm or more from the burr hole.

15. The method of claim 1, wherein the electrode is positioned in white matter or grey matter.

16. The method of claim 15, wherein the electrode is positioned in white matter.

17. The method of claim 4, wherein the catheter comprises the electrode.

18. (canceled)

19. The method of claim 1, wherein generating the AI model based on the electrical signals comprises generating a deep neural network based on the electrical signals.

20. The method of claim 19, further comprising applying the deep neural network to manage the treatment of the subject.

21. (canceled)

22. (canceled)

23. (canceled)

24. The method of claim 1, wherein monitoring electrical signals during the initial recovery phase comprises detecting epileptiform conditions or sleep disorders.

25. The method of claim 1, wherein monitoring electrical signals during the subsequent recovery phase comprises detecting behavioral disturbances.

26. The method of claim 1, wherein monitoring electrical signals comprises continuously monitoring electrical signals.

27. The method of claim 1, wherein modifying the treatment of the subject comprises delivering a rehabilitation intervention based on the AI model.

28. The method of claim 1, wherein modifying the treatment of the subject comprises adjusting a frequency, intensity, timing, or type of a medication.

29. The method of claim 1, wherein treating the subject comprises draining CSF from the CSF-containing space of the subject via the catheter prior to administering a therapeutic.
